(19) United States
(12) Patent Application Publication
(10) Pub. No.: US 2016/0066895 A1
(43) Pub. Date: Mar. 10, 2016

(54) Bodily Fluid Specimen Collection and Assay Device

(71) Applicant: MICROTRI LIMITED, Hong Kong (CN)

(72) Inventors: Bernhard Andreas Schwyn, Chonburi (TH); Glenn Norman Tyler, Chonburi (TH)

(73) Assignee: MICROTRI LIMITED, Hong Kong (CN)

(21) Appl. No.: 14/888,240

(22) PCT Filed: Apr. 25, 2014
(86) PCT No.: PCT/IB2014/000755

§ 371 Date: Oct. 30, 2015

Related U.S. Application Data
(60) Provisional application No. 61/817,414, filed on Apr. 30, 2013.

(51) Int. Cl. A61B 10/00

(52) U.S. Cl. CPC A61B 10/0045; A61B 2010/0009; A61B 2019/446

(57) ABSTRACT

The present invention is directed to a bodily fluid specimen collection and assay device having a bodily fluid collection cup and an integral hinged lid connected to the bodily fluid collection cup. The integral hinged lid forms a seal with the bodily fluid collection cup. The bodily fluid collection cup has a main collection chamber, an assaying test strip viewing window, a test strip magazine, a magazine supporting member, and assaying test strip. The flow of the bodily fluid specimen from the main collection chamber is controlled by plurality of flow ports and outward flow ports along with damming rib and raised rib.

### TECHNICAL FIELD

This invention relates to a bodily fluid specimen collection and assay device.

### BACKGROUND OF THE INVENTION

The practice of testing bodily fluid specimens for health abnormalities and or evidence of the consumption of a number of illicit and illegal drugs has seen the development of a variety of specimen collection and assay devices for use at home, in the workplace, hospitals, law enforcement authorities, sporting arenas and more recently at social gathering places. These devices can be used for a number of purposes including the detection of illicit drugs such as cocaine, heroin, amphetamine, methamphetamines, morphine, marijuana, opiates, THC, AMP, PCP, sporting performance enhancement drugs and hormones. Many of the testing devices for these drugs are complex in design and difficult to use. They are manufactured using multiple grade raw materials creating disposal and recycling difficulties. Furthermore, they are expensive to produce and do not offer total tamper proof security.

PCT/US2004/038428 disclosed a sample collection cup with integrated sample analysis system where the chamber with strips for testing is contained in the lid. The lid is separate from the sample collection cup. When depositing the bodily fluid into the cup, the user must find a place to put the lid. This is a burden for the user and potentially cause sample contamination as the lid may become contaminated if placed in an unclean area.

U.S. Pat. No. 8,394,626 B2 disclosed a specimen collection and assay container with a separate lid. The lid is a tamper resistant seal with a series of sloped projections. A separated lid is cumbersome for the user and the series of sloped projections do not visually show if the lid has been tampered with. A more practical specimen collection and assay device with better tampering prevention or better tampered-evident is desirable.

### SUMMARY OF THE INVENTION

The present invention is directed to a bodily fluid specimen collection and assay device having a bodily fluid collection cup comprising a bottom and a surrounding wall defining a main collection chamber and an assaying test strip viewing window define on the said wall of the bodily fluid collection cup comprising a damming rib. The bodily fluid specimen collection and assay device has an integral hinged lid integrally hinged to the said bodily fluid collection cup, a magazine supporting member disposed within the main collection chamber. The said magazine supporting member comprises a plurality of flow ports disposed at the lower section of magazine supporting member. The bodily fluid specimen collection and assay device has a test strip magazine comprising: a plurality of recessed slots each of which is configured to accommodate an assaying test strip, a damming device disposed on each recessed slot, an outward flow port disposed on each damming device; disposed at a position higher than the position of the flow ports on the magazine supporting member; and a raised rib positioned to match the height of the damming rib. The damming rib, the raised rib, the test strip magazine and the magazine supporting member are engaged to create differential pressure chamber.

In an embodiment, the bodily fluid specimen collection and assay device and the integral hinged lid are prepared with corresponding coupling element configured to provide leak proof permanent seal to the bodily fluid specimen collection and assay device.

In another embodiment, the bodily fluid specimen collection and assay device has an extraction port prepared on the integral hinged lid.

In yet another embodiment, the bodily fluid specimen collection and assay device comprises a drainage port prepared on the bottom of the main collection chamber of the bodily fluid collection cup.

In an embodiment, the bodily fluid specimen collection and assay device comprises an extraction port prepared on the integral hinged lid and a drainage port prepared on the bottom of the main collection chamber of the bodily fluid collection cup.

In yet another embodiment, the bodily fluid specimen collection and assay device comprises an integral hinged lid with a protruding rib support along top of the test strip magazine.

In an embodiment, the bodily fluid specimen collection and assay device comprises an integral hinged lid with a protruding rib configured to rest on the protruding rib support on the integral hinged lid as the integral hinged lid is coupled to the bodily fluid collection cup.

In another embodiment, the bodily fluid specimen collection and assay device comprises recessed slot of a shape corresponding to a shape of a test strip.

In an embodiment, the bodily fluid specimen collection and assay device comprises test strip magazine with up to eight recessed slots.

In another embodiment, the bodily fluid specimen collection and assay device comprises at least an area for identification labeling.

In an embodiment, the bodily fluid specimen collection and assay device comprises an extraction port comprising a self-resealed polymeric material or membrane.

### BRIEF DESCRIPTION OF THE DRAWINGS

Advantages and characteristics of the invention will be appreciated from the following description, in which, as a non-limiting example, some preferable embodiments of the principle of the invention are described, with reference to the accompanying drawings, in which:

FIG. 1 illustrates a perspective view of an embodiment of the bodily fluid specimen collection and assay device showing the hinged lid open;

FIG. 2 illustrates a sectional perspective view of an embodiment of the bodily fluid specimen collection and assay device showing the hinged lid open; and a drainage port;

FIG. 3 illustrates an embodiment of the bodily fluid specimen collection and assay device hinged lid closed; and an extraction port defined on the hinged lid;

FIG. 4 illustrates a scrap sectional view of the embodiment of the bodily fluid specimen collection and assay device of FIG. 3 and its corresponding coupling element with coupling profiles between the bodily fluid collection cup and the integral hinged lid;

FIG. 5 illustrates an embodiment of front of a test strip magazine;

FIG. 6 illustrates an embodiment of back of a test strip magazine;

FIG. 7 illustrates a sectional perspective view of an embodiment of the bodily fluid specimen collection and assay device with the hinged lid closed and showing the outward flow port of the damming device in relation to the flow port of the magazine supporting member;

FIG. 8 illustrates a scrap sectional view of the embodiment of FIG. 7 illustrating direction of flow of the bodily fluid specimen through the test strip magazine;

FIG. 9 illustrates a scrap sectional view of the embodiment of FIG. 7 illustrating sample filled bodily fluid specimen collection and assay device and flow of the bodily fluid specimen through the test strip magazine; and

FIG. 10 illustrates an embodiment of the bodily fluid specimen collection and assay device illustrating an assaying test strip viewing window with assaying test strips placed within the bodily fluid specimen collection and assaying device, assaying test strip conjugate zone and reaction zone.

### DETAILED DESCRIPTION OF PREFERRED EMBODIMENT OF THE INVENTION

The present invention is directed to a bodily fluid specimen collection and assay device having a bodily fluid collection cup comprising a bottom and a surrounding wall defining a main collection chamber and an assaying test strip viewing window define on the said wall of the bodily fluid collection cup comprising a damming rib. The bodily fluid specimen collection and assay device has an integral hinged lid integrally hinged to the said bodily fluid collection cup, a magazine supporting member disposed within the main collection chamber. The said magazine supporting member comprises a plurality of flow ports disposed at the lower section of magazine supporting member. The bodily fluid specimen collection and assay device has a test strip magazine comprising: a plurality of recessed slots each of which is configured to accommodate an assaying test strip, a damming device disposed on each recessed slot, an outward flow port disposed on each damming device; disposed at a position higher than the position of the flow ports on the magazine supporting member; and a raised rib positioned to match the height of the damming rib. The damming rib, the raised rib, the test strip magazine and the magazine supporting member are engaged to create differential pressure chamber.

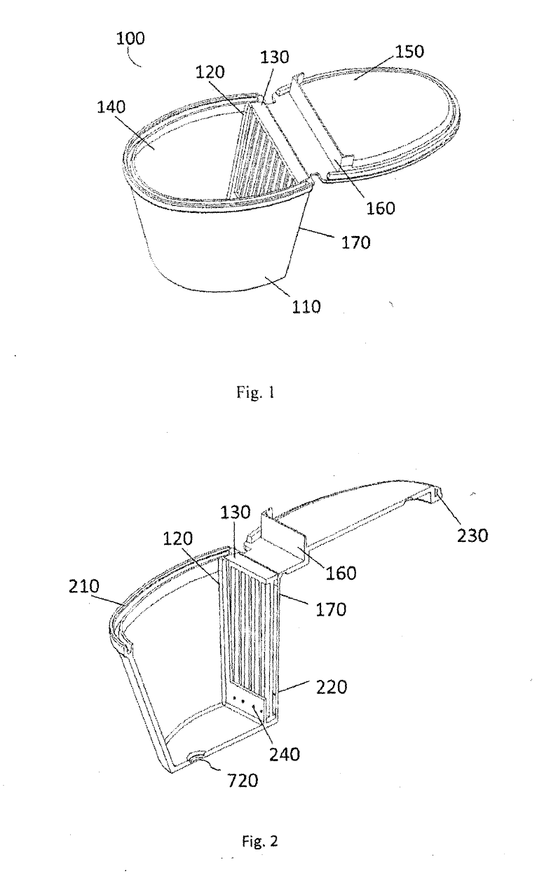

FIGS. 1-10 show a bodily fluid specimen collection and assay device 100 according to the principle of the invention.

In more detail, FIG. 1 illustrates an embodiment of the bodily fluid specimen collection and assay device 100. The bodily fluid specimen collection and assay device 100 will now be referred to as “device.” The device 100 comprises a bodily fluid collection cup 110 and an integral hinged lid 150 integrally hinged to the bodily fluid collection cup 110. The bodily fluid collection cup 110 will now be referred to as the “collection cup.” The collection cup 110 comprises a bottom wall with a surrounding wall to define a main collection chamber 140. The said wall defines an assaying test strip viewing window 170 to facilitate viewing of the test results which will be further described below. The collection cup 110 also comprises a magazine supporting member 120 disposed within the main collection chamber 140 at a corresponding position at which the assaying test strip viewing window 170 is located, such that a pocket between the wall of the collection cup 110 which is designated as the assaying test strip viewing window 170 and the magazine supporting member 120 is formed so as to accommodate a test strip magazine 130 to be inserted therein. Both collection cup 110 and the integral hinged lid 150 are prepared with a corresponding coupling element such that a leak proof permanent seal is achieved once the integral hinged lid is engaged with the collection cup 110. Further, the integral hinged lid 150 is not only configured to securely couple with the collection cup 110 so as to contain a sample, but also functioning as a handle during the act of depositing a bodily fluid specimen such as urine into the collection cup 110. The integral hinged lid 150 retains its post-molded rigidity and is able to support a maximum of 100 grams of bodily fluid specimen deposited within the collection cup 110, and preferably without flexing and causing possible spillage of the deposited bodily fluid specimen. The hinged feature of the integral hinged lid 150 allows the integral hinged lid 150 to be pivoted into position over and above the collection cup 110 and snap-fastened with the collection cup 110 through the corresponding coupling element prepared on both the collection cup 110 and the integral hinged lid 150 mentioned earlier.

In an embodiment, the integral hinged lid 150 comprising a protruding rib 160 located in proximity to the hinged section thereof. The protruding rib 160 rests on the protruding rib support when the integral hinged lid is closed 150. The protruding rib 160 is configured to interact with the test strip magazine 130 situated inside the collection cup 110 such that once the integral hinged lid 150 is closed the protruding rib 160 secures the test strip magazine 130 in place.

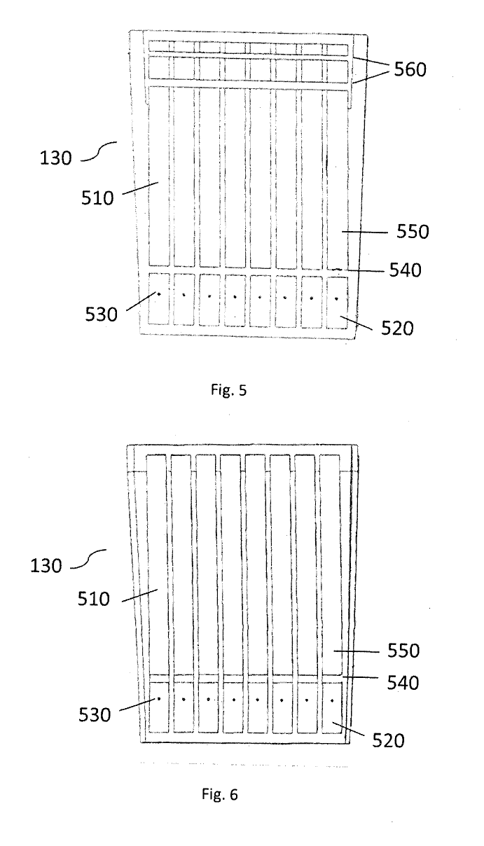

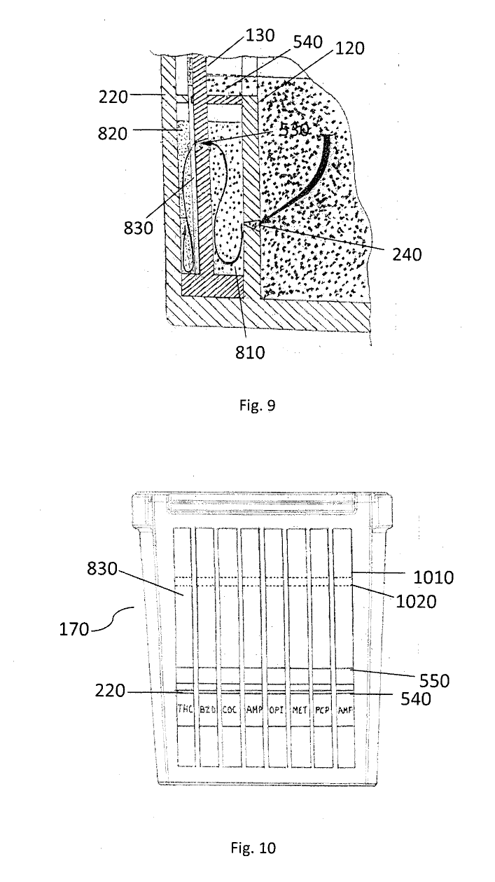

FIG. 2 illustrates a sectional perspective view of an embodiment of the device 100. In this embodiment, the device 100 comprises a drainage port, the detail of which will be further described. Also illustrated in FIG. 2 is the test strip magazine 130 configured to fit tightly between the test strip viewing window 170 and the magazine supporting member 120. The magazine supporting member 120 comprises a plurality of flow ports 240 disposed at the lower section of the magazine supporting member 120. The numbers of the flow port 240 correspond to the number of recessed slots 510 prepared on the test strip magazine 130, the detail of which will be further described. It is essential that each of the flow port 240 of the magazine supporting member 120 is located lower than the height of an outward flow port 530 prepared on the test strip magazine 130 and that it is also essential that the outward flow port 530, must be located lower than the height of a damming rib 220 prepared on the wall of the collection cup 110 at which the assaying viewing window 170 is located. Further, the damming rib 220 is situated, preferably less than half the height of the assaying test strip viewing window 170 to match the height of the raised rib 540 (shown in FIG. 5). Once the test strip magazine 130 is inserted into the space between the assaying viewing window 170 and the magazine supporting member 120, the damming rib 220, the raised rib 540, the test strip magazine 130, and the magazine supporting member 120 together creates a mechanism to slow down the flow of the bodily fluid into the assaying test strip 830, see FIGS. 8 and 9.

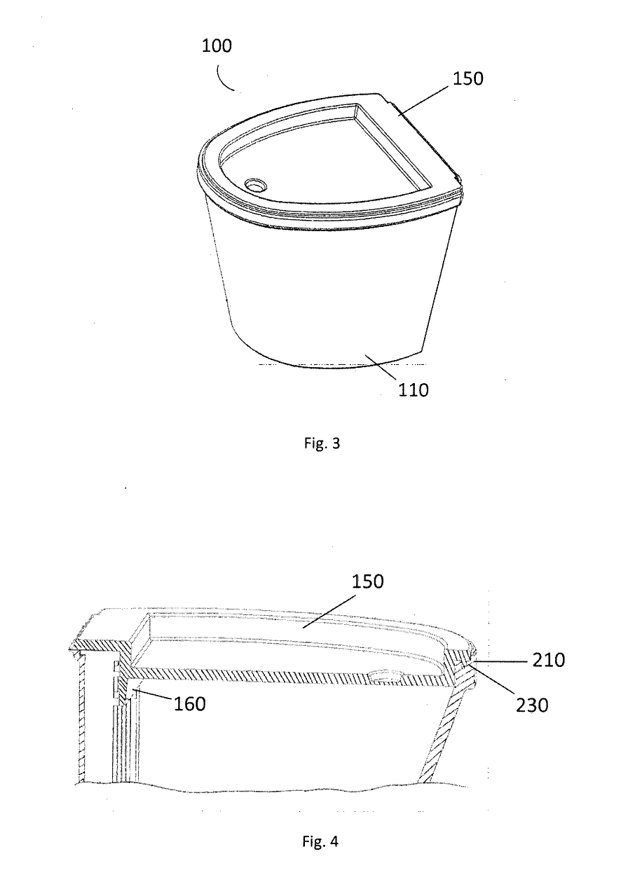

As mentioned earlier that both the collection cup 110 and the integral hinged lid 150 are prepared with corresponding coupling elements in order to provide a leak proof permanent seal to the device 100, in an embodiment, the mentioned coupling elements are realized as, for example, the collection cup 110 comprises a groove 210 along an edge with a raised rib profile. The integral hinged lid 150 comprises a tongue 230 surrounding the edge of the integral hinged lid 150. The tongue 230 is configured to be compatible with the groove 210 on the edge of the collection cup 110 such that coupling between them provides a tight leak proof permanent seal of the device 100 as illustrated in FIGS. 3 and 4.

After a bodily fluid specimen is deposited into the collection cup 110, the integral hinged lid 150 is pivoted downward to engage with the collection cup 110 so as to close the device 100. The coupling between the coupling element on the collection cup 110 and the integral hinged lid 150 provides a leak proof permanent seal to the device 100. Once the device 100 is closed and as the coupling is intended to provide a permanent seal to the device 100 re-opening of the device 100 without force is near impossible. If force is used to pry open the integral hinged lid 150 there is a good chance that there will be visual evidence (damages or deformation) to the device 100 suggesting that the sample may have been tampered with. Further, if the device 100 is submitted to stress or bending it will form a white section known as stress whitening. This reduces the risk of tampering or cross contamination of sample.

Further, as mentioned above, the integral hinged lid 150 also comprises the protruding rib 160 of which upon closing of the integral hinged lid 150, the protruding rib 160 abut against the test strip magazine 130. By such arrangement, the protruding rib 160 not only helps to secure the test strip magazine in place, but also provide an added seal to the device 100. In more detail, the protruding rib 160 is located across the top of the test strip magazine 130. The presence of the protruding rib 160 avoids the need for additional sealing membranes such as O-rings or additional seals to achieve a dynamic leak proof condition.

As illustrated in FIG. 5, there is shown front of the test strip magazine 130 detailing the recessed slots 510. The front of the test strip magazine 130 is facing the main collection chamber 140. The test strip magazine 130 is configured to be fitted between the magazine supporting member 120 and the assaying test strip viewing window 170.

As mentioned above, the test strip magazine 130 comprises a plurality of recessed slots 510. Each recessed slot 510 is configured to accommodate an assaying test strip 830 to be inserted to the said recessed slots 510. The shape and configuration of the recessed slots 510 should correspond with the shape of the assaying test strip 830. In an exemplary embodiment, the recessed slot 510 is rectangular in shape so as to accommodate a rectangular assaying test strip 830.

Each recessed slot 510 comprises a damming device 520 each of which comprises an outward flow port 530. Above the damming device 520 facing the main collection chamber 140 is a raised rib 540 which forms part of the damming device 520. The damming device 520 is configured to serve the function of limiting the amount of bodily fluid specimen within and around the lower portion of the assaying test strips 830 (see FIG. 9). The raised rib 540 is preferably positioned below a conjugate zone 550 of the assaying test strip 830, which is below the middle portion of the assaying test strip 830 to keep the level of bodily fluid below the conjugate zone 550. The test strip conjugate zone 550 is where the reactive element takes place. Within the conjugate zone 550 there are a number of chemicals pre-coated with drug-protein conjugate for testing the presence of drugs in the bodily fluid, Thus failure to retain the flow level of the bodily fluid specimen well below the conjugate zone 550 may render the assaying test strip 830 ineffective or resulting in inaccurate or inconclusive or false test results. The test strip magazine 130 comprises of a protruding rib support 560 near the top facing the main collection chamber 140 whereupon the hinged lid 150 is closed, the protruding rib 160 rests on the protruding rib support 560.

FIG. 6 illustrates back of the test strip magazine 130. The back of the test strip magazine 130 is facing the assaying test strip viewing window 170. The recessed slots 510 with the damming device 520 are shown. The recessed slot 510 is dimensioned to correspond with the dimensions and configurations of the assaying test strip 830 such that each recessed slot 510 can accommodate an assaying test strip 830. Preferably, each assaying test strip 830 is nested vertically into each individual recessed slot 510. The assaying test strip 830 may be fitted by hand or by robotic machine in a clean room and zero humidity environment. The test strip magazine 130 may be preloaded with one or more assaying test strips 830. In an embodiment, there are up to eight recessed slots 510 with eight assaying test strips 830 for testing wherein each assaying test strip is designated to be reactive to one specific substance or as an indicator of presence of such substance. This means that an array of different substances may be tested for, with only a single test using a single sufficient sample of specimen. The assaying test strip 830 is preferably made of an absorbent material and has a conjugate zone 550 that is positioned such that it allows the specimen to come into contact with or reacted with the assaying test strip 830. In an embodiment, the conjugate zone 550 is positioned below the middle of the assaying test strip 830.

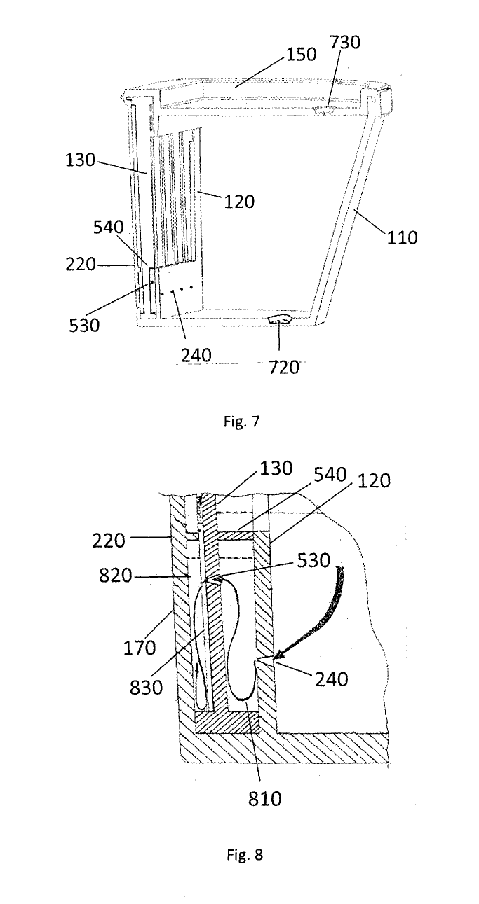

FIG. 7 illustrates the sectional perspective view of an embodiment of the device 100 in a fully closed state. As shown, the magazine supporting member 120 comprises a plurality of flow port 240 wherein the said plurality of flow ports 240 disposed at the lower section of the magazine supporting member 120. The numbers of the flow port 240 correspond to the number of recessed slots 510 prepared on the test strip magazine 130, and each flow port is located a position corresponding to each recessed slot 510. The flow port 240 is situated lower than the outward flow port 530 of the damming device 520. This is to restrict the amount and pace of the fluid that is flowing towards the assaying test strip 830 as too much bodily fluid contacting the assaying test strip 380 at one time may result in false positive. The test strip magazine 130 has raised rib 540 in the front side facing the main collection chamber 140 and the raised rib 540 is prepared with the same height as the damming rib 220. The raised rib 540 and the damming rib 220 together create a sealing effect so as to further reduce the bodily fluid pressure slowing down the flow of the bodily fluid. The raised rib 540 and damming rib 220 are dimensioned to correspond to one another such that they sufficiently and effectively encompass the assaying test strips 830 without having any adverse effects on or interfere with or restrict the capacity of the assaying test strip. For example, they must not squeeze the assaying test strips 830 too tightly such that it could restrict capillary fluid flow through the strips.

The main collection chamber 140 is configured to contain a volume of bodily fluid sample. In an embodiment, the collection chamber is capable of receiving up to 100 milliliters of bodily fluid specimen. In an embodiment, the bodily fluid specimen collection and assay device 100 comprising a drainage port 720 prepared at a main collection chamber 140 of the collection cup 110. In an embodiment as shown in FIGS. 2 and. 7, the drainage port 720 is prepared on the bottom region of the main collection chamber 140. The drainage port 720 is provided as a practical means for discharging of the bodily fluid specimen from the main collection chamber 140 for disposal and or recycling purposes. In an embodiment, the drainage port 720 may be constructed from materials made to be easily penetrated with a suitable tool such that the fluid flowing may be discharged through the port and safely disposed of.

Further, in an embodiment, the device 100 comprising an extraction port 730, disposed, preferably on the integral hinged lid 150. As shown in FIGS. 3 and 7, the integral hinged lid 150 comprises an extraction port 730 to permit extraction or retrieval of the sample contain inside the main collection chamber 140 without the need to unseal the hinged lid 150 from the collection cup 110. As an example, the extraction port 730 may be made of self-resealed polymeric materials or membrane such that a sample of bodily fluid may be easily and safely extracted from the main collection chamber 140 using a suitable tool such as a hypodermic needle wherein the needle entry point is reseal upon withdrawal of the needle. With this characteristic, a secondary fluid specimen testing and analysis can be conducted without disengaging the sealed bodily fluid specimen collection and assay device 100.

FIG. 8 illustrates the direction of the flow of the bodily fluid specimen from a main collection chamber 140 through the flow ports 240 into a pressure reduction chamber 810, then through the outward flow ports 530 into an assaying test strip chamber 820 in a controlled hydraulic and preferred manner. The pressure reduction chamber 810 is formed as a result of engagement of the damming device 520, the raised rib 540 and the magazine supporting member 120. The assaying test strip chamber 820 is formed as a result of engagement of the assaying test strip viewing window 170, the damming rib 220 and the test strip magazine 130.

The damming device 520, raised rib 540, damming rib 220, magazine supporting member 120, flow port 240, and outward flow port 530 are arranged so as to gradually reduce the fluid head pressure during the flow of the provided bodily fluid specimen from the main specimen collection chamber by restricting the flow of the bodily fluid specimen into a pressure reduction chamber 810 establishing a lesser fluid head pressure within the pressure reduction chamber 810. The speed of fluid transfer flow from the main collection chamber 140 to the pressure reduction chamber 810 can be controlled by increasing or decreasing the flow port 240 diameter. This is to adjust the pressure so that the fluid does not contact the assaying test strip 830 too fast resulting in false result.

FIG. 9 illustrates a controlled flow of the provided bodily fluid specimen depicted by the flow directional arrow. Bodily fluid specimen is deposited into the main collection chamber 140 and the bodily fluid specimen moves into the pressure reduction chamber 810 via the flow ports 240 disposed on the magazine supporting member 120. The bodily fluid specimen then flows from the pressure reduction chamber 810 into the assaying test strip chamber 820 very slowly via the outward flow ports 530 disposed on the test strip magazine 130. The bodily fluid specimen seeps into the assaying test strip 830 and the test procedure commences. The damming rib 220 and the raised rib 540 are positioned adjacent to each other when the test strip magazine 130 is fitted into the magazine supporting member 120. The damming rib 220 and the raised rib 540 provide pressure laterally and on either side of the assaying test strips 830. Both the damming rib 220 and the raised rib 540 are located below the assaying test strip conjugate zone 550. The damming rib 220 and the raised rib 540 ensure that the bodily fluid specimen entering the assaying test strip chamber 820 only reaches the preferred operable fluid level and in relation to the positioning of the assaying test strip conjugate zone 550.

As mentioned above, the bodily fluid collection cup 110 comprises a bottom with a surrounding wall to define a main collection chamber 140. The said wall defines an assaying test strip viewing window 170 to facilitate viewing of the test results. FIG. 10 illustrates the bodily fluid collection cup 110 with the assaying test strip viewing window 170 having assaying test strips 830 positioned in place and within the test strip magazine 130. The assaying test strip viewing window 170 is transparent so that test result can be viewed through the assaying test strip viewing window 170 without having to open the bodily fluid specimen collection and assay device 100. The assaying test strip viewing window 170 and the assaying test strips 830 are clearly visible when the device is placed on its base on a flat surface such as a table.

As mentioned, the assaying test strip viewing window 170 comprises a damming rib 220. The preferred position of the damming rib 220 and the raised rib 540 are shown in relation to the assaying test strip conjugate zone 550 and the assaying test strip control region 1010 and test region 1020, which indicates the test results. The bodily fluid specimen collection and assay device 100 is made by injection molding where the bodily fluid collection cup 110 and the integral hinged lid 150 are produced as one piece. The bodily fluid specimen collection and assay device 100 is made of polymer. The bodily fluid specimen collection and assay device 100 is produced using a common injection molding grade of plastic resin, for example Random Copolymer Polypropylene. Thus the material is quite soft. If a hand tool is used to open the integral hinged lid 150 there will be visual evidence. The plastic whitens if submitted to stress or bending. The whitening of the plastic is a good indicator that the bodily fluid specimen collection and assay device 100 has been tampered with.

The bodily fluid specimen collection and assay device 100 comprises at least an area for identification labeling. The bodily fluid specimen collection and assay device 100 can be labeled using a writing instrument or by etching. The etching is useful for labeling the subject's identification and the name of specific assays, at the same time eliminating the need for expensive adhesive labels and other decorative components. The bodily fluid specimen collection and assay device 100 comprises a shape of a standard toilet bowl and lid.

The present invention is more cost effective and less complicated to manufacture when compared to other known and commercially available bodily fluid specimen collection devices. The bodily fluid specimen collection and assay device 100 is in one piece, convenient and easy to use.

1. A bodily fluid specimen collection and assay device comprising:
a bodily fluid collection cup comprising a bottom and a surrounding wall defining a main collection chamber and an assaying test strip viewing window define on the wall of the bodily fluid collection cup comprising a damming rib;
an integral hinged lid integrally hinged to the bodily fluid collection cup;
a magazine supporting member disposed within the main collection chamber, the magazine supporting member comprising a plurality of flow ports disposed at the lower section of magazine supporting member; and
a test strip magazine comprising:
a plurality of recessed slots each of which is configured to accommodate an assaying test strip;
a damming device disposed on each recessed slot;
an outward flow port disposed on each damming device; disposed at a position higher than the position of the flow ports on the magazine supporting member; and
a raised rib positioned to match the height of the damming rib;
wherein the damming rib, the raised rib, the test strip magazine and the magazine supporting member engage to create a differential pressure chamber.

2. The bodily fluid specimen collection and assay device of claim 1, wherein the bodily fluid collection cup and the integral hinged lid are prepared with a corresponding coupling element configured to provide leak proof permanent seal to the bodily fluid specimen collection and assay device.

3. The bodily fluid specimen collection and assay device of claim 2, further comprising an extraction port prepared on the integral hinged lid.

4. The bodily fluid specimen collection and assay device of claim 2, further comprising a drainage port prepared on the bottom of the main collection chamber of the bodily fluid collection cup.

5. The bodily fluid specimen collection and assay device of claim 3, further comprising a drainage port prepared on the bottom of the main collection chamber of the bodily fluid collection cup.

6. The bodily fluid specimen collection and assay device of claim 2, wherein the integral hinged lid further comprises a protruding rib support along top of the test strip magazine.

7. The bodily fluid specimen collection and assay device of claim 6, wherein the integral hinged lid further comprises a protruding rib configured to rest on the protruding rib support on the integral hinged lid as the integral hinged lid is coupled to the bodily fluid collection cup.

8. The bodily fluid specimen collection and assay device of claim 1, wherein the recessed slot is of a shape corresponding to a shape of a test strip.

9. The bodily fluid specimen collection and assay device of claim 1, wherein the test strip magazine comprises up to eight recessed slots.

10. The bodily fluid specimen collection and assay device of claim 1, comprising at least an area for identification labeling.

11. The bodily fluid specimen collection and assay device of claim 1, comprising an extraction port comprising a self-resealed polymeric material or membrane.

12. The bodily fluid specimen collection and assay device of claim 5, wherein the integral hinged lid further comprises a protruding rib support along a top of the test strip magazine.

13. The bodily fluid specimen collection and assay device of claim 7, wherein the recessed slot is of a shape corresponding to a shape of a test strip.
